(19) United States
(12) Patent Application Publication
(10) Pub. No.: US 2024/0390376 A1
(43) Pub. Date: Nov. 28, 2024

(54) METHOD FOR PREVENTING ADVERSE PREGNANCY OUTCOMES DUE TO USE OF SELECTIVE SEROTONIN REUPTAKE INHIBITORS (SSRI) DURING GESTATION

(71) Applicant: Wisconsin Alumni Research Foundation, Madison, WI (US)

(72) Inventors: Laura Hernandez, Middleton, WI (US); Rafael Reis Domingues, Madison, WI (US); Milo Wiltbank, Verona, WI (US)

(73) Assignee: Wisconsin Alumni Research Foundation, Madison, WI (US)

(21) Appl. No.: 18/671,308

(22) Filed: May 22, 2024

Related U.S. Application Data
(60) Provisional application No. 63/503,637, filed on May 22, 2023.

(51) Int. Cl. A61K 31/517; A61P 15/06

(52) U.S. Cl. CPC A61K 31/517; A61P 15/06

(57) ABSTRACT

A method to inhibit adverse pregnancy outcomes in a pregnant mammal via administering to the pregnant mammal who is concurrently taking a selective serotonin reuptake inhibitor (SSRI) a therapeutically effective amount of a serotonin receptor antagonist.

### CROSS-REFERENCE TO RELATED APPLICATIONS

Priority is hereby claimed to provisional application Ser. No. 63/503,637, filed May 22, 2023, which is incorporated herein by reference.

### FEDERAL FUNDING STATEMENT

This invention was made with government support under HD094759 awarded by the National Institutes of Health. The government has certain rights in the invention.

### BACKGROUND

Selective serotonin reuptake inhibitors (SSRIs) encompass a class of psychotropic medications used to treat several psychological conditions including depression, obsessive compulsive disorder, and panic disorder in adult and pediatric patients. (Benfield et al. Drugs. 1986; 32 (6): 481-508.). Fluoxetine, the first SSRI introduced to the U.S. market, has been commercially available since 1987. Since then, other SSRIs have become available and now eight compounds have been approved by the FDA for patient use. Among these, sertraline, fluoxetine, and citalopram are the most commonly prescribed to pregnant women. (Bandoli et al. Pediatrics. 2020; 146 (1).) Because the use of SSRIs, particularly by women during reproductive age, has dramatically increased over the last three decades, numerous women are exposed to SSRIs during gestation. (Koren G, Nordeng H. Am J Obstet Gynecol. 2012; 207 (3): 157-63. Cooper et al. Am J Obstet Gynecol. 2007; 196 (6): 544. e1-. e5.) The frequency of pregnant women taking SSRIs during gestation varies from 6 to 13% among studies. Approximately 300,000 women and their infants yearly in the USA are exposed to SSRIs during a critical period that is determinant for adequate maternal and fetal/neonatal wellbeing. In a recent report, 92.2% of women that took an SSRI during gestation were using the SSRI when they became pregnant and only about 38% of women discontinued treatment by week 13 of gestation (Bandoli et al., supra).

For decades, SSRIs have been associated with adverse pregnancy outcomes in humans and animal models. It has been reported that up to 30% of infants may display some clinical manifestation related to maternal SSRI use. (Belik J. Semin Perinatol. 2008; 32 (5): 350-4.) Adverse pregnancy outcomes most frequently associated with maternal SSRI use and encountered more often by clinicians are decreased birthweight/small for gestational age and preterm birth. Nevertheless, dozens of other pregnancy and neonatal adverse effects have been reported, including postpartum hemorrhage, birth defects, persistent pulmonary hypertension of the newborn, neonatal cardiac issues, increased NICU admissions, neonatal abstinence/toxicity, postnatal adaptation syndrome, developmental delays, autism spectrum disorder, neonatal jitteriness, increased hospital admission up to two years of birth, neonatal death, seizures, endocrine disruption, infant obesity, and respiratory distress. (Colvin et al. CNS Drugs. 2012; 26 (7): e1-e14. Hale et al. Clinical pediatrics. 2001; 40 (12): 681-4. Alchan et al. The Journal of Maternal-Fetal & Neonatal Medicine. 2008; 21 (12): 921-3. Laine et al. Arch Gen Psychiatry. 2003; 60 (7): 720-6. Sivojelezova et al. Am J Obstet Gynecol. 2005; 193 (6): 2004-9. Berard et al. Br J Clin Pharmacol. 2016; 81 (4): 589-604. Bérard A, Zhao J-P, Shechy O. BMJ open. 2017; 7 (1): c013372. Davidson et al. Pediatr Res. 2009; 65 (2): 236-41. El Marroun et al. Arch Gen Psychiatry. 2012; 69 (7): 706-14. Grzeskowiak et al. J. Dev. Origins of Health and Disease. 2012; 3 (4): 253-61. Norby et al. Pediatrics. 2016; 138 (5). Borue et al. Int's J. Dev. Neuroscience. 2007; 25 (6): 341-7.)

Numerous reports have investigated the role of maternal SSRI use on the occurrence of persistent pulmonary hypertension of the newborn as it appears to be the one of the main neonatal side effects related to in utero exposure to a SSRI. (Chambers et al. Selective serotonin-reuptake inhibitors and risk of persistent pulmonary hypertension of the newborn. N Engl J Med. 2006; 354 (6): 579-87. Ng et al. J. of Women's Health. 2019; 28 (3): 331-8.)

Although the pharmacokinetics of different SSRIs vary substantially, all of them inhibit serotonin transporter (SERT). (DeVane C L. Metabolism and pharmacokinetics of selective serotonin reuptake inhibitors. Cell Mol Neurobiol. 1999; 19 (4): 443-66.) Inhibition of SERT in the central nervous system culminates with its psychotropic actions. (Harmer et al. Philosophical Transactions of the Royal Society B: Biological Sciences. 2013; 368 (1615): 20120407. Liu et al. Front Cell Neurosci. 2017; 11:305.). However, in addition to its role in the brain, SSRIs also inhibit peripheral SERT-modulating serotonin signaling throughout the body. (Fouquet et al. Pharmacol Res. 2019; 140:67-74.) Inhibition of SERT on peripheral tissues prevents serotonin transport into the cell for degradation, thereby increasing serotonin signaling through its cell surface receptors. Furthermore, inhibition of SERT on platelets prevents the platelets' ability to uptake free serotonin in the blood. (Blardi et al. J Clin Psychopharmacol. 2002; 22 (2): 131-6.)

Levy et al., J Perinatol. 2020; 40 (7): 1017-24, reported multiple placental pathologies in women undergoing SSRI treatment during gestation. Placental vascular lesions of maternal malperfusion were increased 7.4-fold, fetal vascular lesions consistent with fetal thrombo-occlusive disease were increased 3.6-fold, and composite fetal vascular malperfusion lesions were increased 2.4-fold in women taking a SSRI during gestation compared to an untreated control group. Two studies in sheep reported placental alterations consistent with Levy et al.'s results in humans. (Morrison et al. Pediatr Res. 2002; 51 (4): 433-42 and Domingues et al. Frontiers in Medicine. 2022:2358.) Similar effects of increased serotonin have been reported in rodents. (Mitchell et al. Biol Reprod. 1983; 28 (4): 830-5. Salas et al. Hypertension. 2007; 50 (4): 773-9. Honey et al. Am J Obstet Gynecol. 1967; 99 (2): 250-7. Van Cauteren et al. Drug Dev Res. 1986; 8 (1-4): 179-85.)

In third trimester pregnant sheep, fluoxetine rapidly decreased blood perfusion to the uterus which was associated with decreased fetal oxygen saturation, partial pressure of oxygen, and blood pH while partial pressure of carbon dioxide and lactate were increased. (See Morrison et al., supra.) Similarly, in humans, changes in fetal heart rate and brain blood flow at week 36 of gestation associated with maternal SSRI use are consistent with fetal hypoxia. (Rurak et al. Pediatr Res. 2011; 70 (1): 96-101.) In another study, pregnant sheep had decreased placentome growth when treated with fluoxetine during the last month of gestation (Domingues et al., supra). These placental alterations in sheep, and likely in women, appear to be due to fluoxetine inhibition of SERT leading to increased circulating serotonin. The result appears to be vasoconstriction of uteroplacental vessels and ultimately restricted blood perfusion to the uterus and placenta. Other authors have also suggested a similar mechanism of action (and associated adverse pregnancy outcomes). (Morrison et al., Domingues et al., and Rurak et al., supra.)

In animal models, decreased pregnancy rates, shorter gestation length, decreased neonatal weight, and increased neonatal mortality have been reported when dams are treated with an SSRI (See Domingues et al., supra, as well as Da-Silva et al. Brazilian J. Med. and Biol. Res. 1999; 32:93-8. Cabrera et al. Birth Defects Res. 2020; 112 (13): 1014-24. Noorlander et al. PLOS One. 2008; 3 (7). Domingues et al. Toxics. 2022; 10 (1): 11. Muller et al. Basic Clin Pharmacol Toxicol. 2013; 113 (2): 132-40.). The reports of these adverse pregnancy outcomes in different animal models, rodent strains, SSRI drugs, dosages, and periods of exposure strengthen the association between maternal SSRI treatment and unfavorable pregnancy outcomes.

It has recently been shown that fluoxetine decreases pregnancy rates. Both fluoxetine and sertraline decrease the number of pups born and increase pup mortality. (Domingues et al. Toxics. 2022; 10 (1): 11.) Sertraline has limited placental transfer resulting in low concentrations of sertraline in the fetal circulation. (Fouquet et al. and Blardi et al., supra.) Conversely, placental transfer of fluoxetine is about 70%. Nevertheless, both fluoxetine and sertraline decrease the number of pups born and increase neonatal mortality. Researchers have also used sheep to investigate the effects of SSRI on pregnancy and neonatal outcomes (Domingues et al., supra). Fluoxetine treatment during the last month of pregnancy recapitulated several findings associated with SSRI exposure during pregnancy in women (decreased placentome, shorter gestation length, decreased birthweight, neonatal morbidity). The apparent greater impact of SSRI on the occurrence of low birthweight/small for gestational age neonates and preterm birth upon treatment during late pregnancy is consistent with restriction of fetal growth during the period of greater fetal growth during the last trimester of gestation. (Gruenwald et al. Amer. J. Obstetrics & Gynecology. 1966; 94 (8): 1112-9.) The adverse pregnancy outcomes in women, sheep, and rodents highlight the conserved pregnancy effects of altered SERT function and serotonin signaling among species and the biological relevance of these animal models aiming to overcome the effects of SSRI.

### SUMMARY

Selective serotonin reuptake inhibitors (SSRI) are the most common antidepressant used by pregnant women. However, maternal SSRI use has been associated with adverse pregnancy outcomes and perinatal morbidity/mortality in women and animal models. Without being limited to any underlying biological mechanism or phenomenon, the increase in maternal free (plasma) circulating serotonin due to SSRI use is likely the underlying mechanism for decreased blood flow to the uterus, placenta, and fetus. The decreased vascular perfusion limits placental and fetal growth causing placental pathology and increasing the risk for low birthweight/small for gestational age and preterm birth, the main complications associated with SSRI use during gestation. In short, SSRI use in pregnant women decreases blood perfusion to the uterus which results in adverse outcomes on neonatal morbidity and mortality. This is due to activation of serotonin-mediated signaling pathways via the serotonin 2A and 2C receptors causing vasoconstriction of uterine blood vessels. The present disclosure discovers that this signaling pathway can be reversed pharmacologically by administering to a pregnant woman taking an SSRI an effective amount of a selective serotonin receptor antagonist. The preferred serotonin receptor antagonist is ketanserin.

Thus, disclosed and claimed herein is a method to inhibit adverse pregnancy outcomes in a pregnant mammal, comprising administering to the pregnant mammal a therapeutically effective amount of a serotonin receptor antagonist.

The serotonin receptor antagonist is preferably a selective serotonin receptor antagonist. In certain versions, the serotonin receptor antagonist is a 5-HT_{2A/2C} antagonist. Exemplary, non-limiting serotonin receptor antagonists include ketanserin, risperidone, trazodone, and nefazodone. In certain versions, the pregnant mammal is a human.

Also disclosed herein is a method to inhibit adverse pregnancy outcomes in a pregnant mammal, comprising administering to a pregnant mammal who is concurrently taking a selective serotonin reuptake inhibitor (SSRI) a therapeutically effective amount of a serotonin receptor antagonist.

Exemplary, non-limiting SSRIs include racemic citalopram, escitalopram (S-citalopram), fluoxetine, fluvoxamine, paroxetine, and sertraline.

The serotonin receptor antagonist is preferably a selective serotonin receptor antagonist. In certain versions, the serotonin receptor antagonist is a 5-HT_{2A/2C} antagonist. Exemplary, non-limiting serotonin receptor antagonists include ketanserin, risperidone, trazodone, and nefazodone. In certain versions, the pregnant mammal is a human.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G. Effects of fluoxetine on pregnancy outcomes in mice and sheep. FIG. 1A depicts the number of pups born to mice that received no SSRI (control) versus a daily dose of 2 mg/kg and 20 mg/kg fluoxetine. FIG. 1B shows the effect of the same dosages of fluoxetine (2 mg/kg and 20 mg/kg) on the average number of pups weaned. FIG. 1C shows the effect of the same dosages of fluoxetine (2 mg/kg and 20 mg/kg) on mouse pup mortality. FIG. 1D depicts placentome diameter (as a percentage of change from a baseline) in control sheep (no SSRI) and a treatment group given SSRI (fluoxetine) over time. FIG. 1E shows in isolation the placentome diameter ranges at Day 21 in absolute terms (cm). FIG. 1F shows the days of parturition between the control group and the treatment group. FIG. 1G compares lamb birthweights between a treatment group and control group.

FIGS. 2A-2B. Number of pups born and immunostaining for Tunel and caspase 3 (apoptosis) and Ki67 (proliferation) in the placenta (embryonic day 18.5) of mice treated with saline (control), fluoxetine, and fluoxetine+ketanserin. FIG. 2A is a histogram showing the number of mouse pups born in various control and treatment groups (saline, saline+ketanserin, serotonin, serotonin+ketanserin, fluoxetine, and fluoxetine+ketanserinc.) FIG. 2B depicts immunostaining for Tunel and caspase 3 (apoptosis) and Ki67 (proliferation) in the placenta (embryonic day 18.5) of mice treated with saline (control), fluoxetine, and fluoxetine+ketanserin.

FIGS. 3A-3I. Effects of fluoxetine in wild-type mice and serotonin transporter null mice (Sert−/−). FIG. 3A shows the total number of implantation sites (live fetus and resorptions) in wild-type mice (WT) and SERT-null mice (Sert−/−) in control mice (administered saline) and mice administered fluoxetine at 2 mg/kg and 20 mg/kg. FIG. 3B shows the number of live fetuses in the same paired sets of three cohorts: WT control, WT 2 mg/kg SSRI, and WT 20 mg/kg SSRI vs Sert−/− control, Sert−/− 2 mg/kg SSRI, and Sert−/− 20 mg/kg SSRI. FIG. 3C shows embryo/fetal resorptions in six groups. FIG. 3D is a histogram showing pregnancy loss after 10.5 days post coitum (DPC) in wild-type mice and heterozygous Sert+/− mice (as a single cohort) versus SERT-null, Sert−/− mice. FIG. 3E is a histogram showing the number of pups born to the two cohorts. FIG. 3F shows pup birthweight. FIG. 3G is a histogram showing the number of pups weaned in the two cohorts. FIG. 3H records pup mortality within 24 hours in the two groups. FIG. 3I records deaths of entire liters within 24 hours of birth in wild-type and Sert+/− mice (as a single cohort) and Sert−/− mice.

### DETAILED DESCRIPTION

### Abbreviations and Definitions

All references to singular characteristics or limitations of the disclosed method shall include the corresponding plural characteristic or limitation, and vice-versa, unless otherwise specified or clearly implied to the contrary by the context in which the reference is made. The indefinite articles “a” and “an” mean “one or more.”

As used herein, the term “or” is an inclusive “or” operator and is equivalent to the term “and/or” unless the context clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All combinations of method steps disclosed herein can be performed in any order, unless otherwise specified or clearly implied to the contrary by the context in which the referenced combination is made.

The method disclosed herein can comprise, consist of, or consist essentially of the essential elements and steps described herein, as well as any additional or optional ingredients, components, or limitations described herein or otherwise useful in organic chemistry. The disclosure provided herein suitably may be practiced in the absence of any element which is not specifically disclosed herein.

As used herein, the term “serotonin receptor antagonist,” is defined broadly to include any pharmacological agent which inhibits the action of serotonin (or “5-HT”) receptors, now known or developed in the future. “5-HT” is an abbreviation for 5-hydroxytryptamine, the systematic name of serotonin:

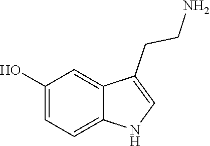 [SMILES: NCCc1c[nH]c2ccc(O)cc12]

### Serotonin (5-hydroxytryptamine)

Explicitly included within the term “serotonin receptor antagonist” are the selective 5-HT_{2A/2C} antagonists ketanserin, risperidone, trazodone, and nefazodone.

Ketanserin is an anti-hypertensive agent having the following structure:

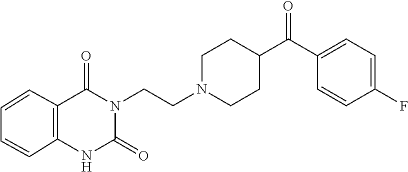 [SMILES: O=C(c1ccc(F)cc1)C1CCN(CCn2c(=O)[nH]c3ccccc3c2=O)CC1]

Regarding its anti-hypertensive effects, see Delaney et al. Amer. J. Physiology—Lung Cellular and Molecular Physiology. 2011; 301 (6): L937-L44. It is typically formulated as hydrochloride or tartrate salt. Its IUPAC systematic name is 3-[2-[4-(4-fluorobenzoyl) piperidin-1-yl]ethyl]-1H-quinazoline-2,4-dione; CAS Nos. 74050-98-9 (for the free base) and 83846-83-7 (for the (+) tartrate salt). It is available commercially under the brand name SUFREXAL® (Johnson & Johnson Corporation, New Brunswick, New Jersey, USA). Tritium-labeled ketanserin hydrochloride is available commercially from RC Tritec Ltd., Appenzell, Switzerland. Ketanserin is a 5-HT_{2A/2C} antagonist.

Risperidone has the following structure:

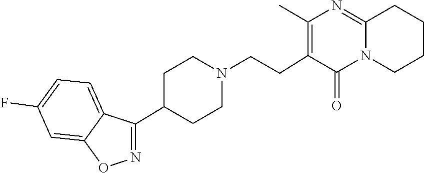 [SMILES: Cc1nc2n(c(=O)c1CCN1CCC(c3noc4cc(F)ccc34)CC1)CCCC2]

It is commercially available in the United States under the brand name RISPERDAL® (Johnson & Johnson Corp., New Brunswick, New Jersey).

Trazadone has the following structure:

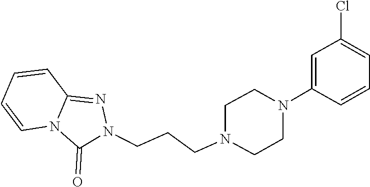 [SMILES: O=c1n(CCCN2CCN(c3cccc(Cl)c3)CC2)nc2ccccn12]

It is commercially available in the United States under the brand name DESYREL® (Pragma Pharmaceuticals, LLC, Locust Valley, New York).

Nefazodone has the following structure:

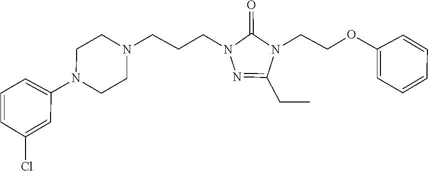 [SMILES: CCc1nn(CCCN2CCN(c3cccc(Cl)c3)CC2)c(=O)n1CCOc1ccccc1]

It is commercially available in the United States as a generic drug (since 2004) from Teva Pharmaceuticals, Ltd. (Tel Aviv, Israel).

IUGR=Intra-uterine growth restriction.

SERT=Serotonin transporter.

SSRI=Selective serotonin reuptake inhibitor. As used herein, SSRI is broadly defined to encompass any selective serotine reuptake inhibitor now known or developed in the future. SSRIs include, but are not limited to, citalopram (e.g., CELEXA®, Forest Laboratories, Inc./H. Lundbeck A/S, New York, New York), escitalopram (e.g., LEXAPRO®, Forest Laboratories, Inc./H. Lundbeck A/S, New York, New York), fluoxetine (e.g., PROZAC®, Eli Lilly and Company, Indianapolis, Indiana), fluvoxamine (e.g., LUVOX®, Abbott Laboratories, Abbott Park, Illinois), paroxetine (e.g., PAXIL®, GlaxoSmithKline LLC, Philadelphia, Pennsylvania), and sertraline (e.g., ZOLOFT®, Viatris Specialy Llc, Morgantown, West Virginia). These compounds are all commercially available.

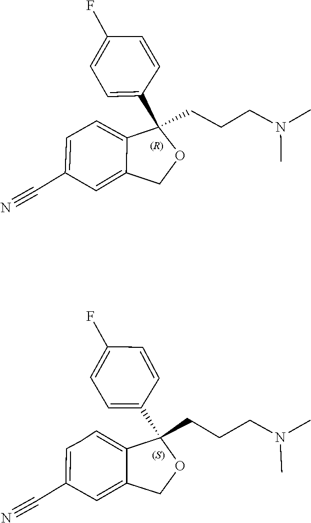

### Citalopram (an R/S Racemic Mixture); Escitalopram (S Enantiomer Only, at Bottom)

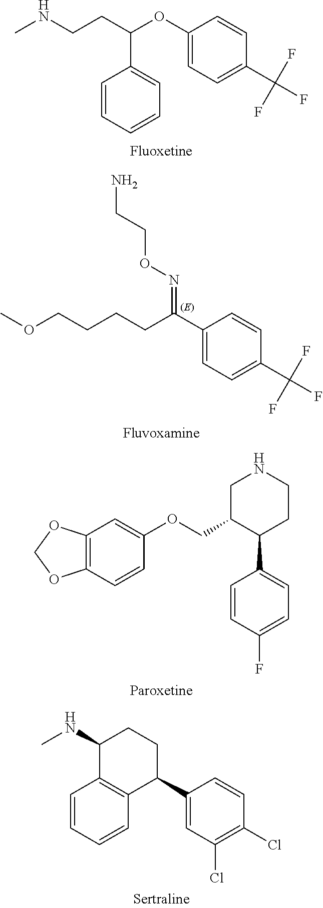 [SMILES: CNCCC(Oc1ccc(C(F)(F)F)cc1)c1ccccc1 ; COCCCC/C(=N\OCCN)c1ccc(C(F)(F)F)cc1 ; Fc1ccc([C@@H]2CCNC[C@H]2COc2ccc3c(c2)OCO3)cc1 ; CN[C@H]1CC[C@@H](c2ccc(Cl)c(Cl)c2)c2ccccc21]

The terms “antagonist” and “inhibitor” are used interchangeably, and they refer to a compound or agent having the ability to inhibit a biological function of a target protein or polypeptide, such as by inhibiting the activity or expression of the target protein, polypeptide, or receptor. Accordingly, the terms “antagonist” and “inhibitor” are defined in the context of the biological role of the target protein, polypeptide, or receptor. While some antagonists herein specifically interact with (e.g., bind to) the target receptors (i.e., they are selective serotonin receptor antagonists), compounds that inhibit the biological activity of the target receptors by interacting with other members of the signal transduction pathway of which the receptors are a part are also specifically included within this definition.

The term “effective amount” or “therapeutically effective amount” refers to that amount of a compound or pharmaceutical composition described herein that is sufficient to affect the intended application, namely selective serotonin receptor antagonism. The therapeutically effective amount can vary depending upon, for example, the weight and age of the subject, the severity of the depression or other mental health condition suffered by the subject, the type of SSRI used by the subject, the length of time the SSRI has been taken, etc., all of which can readily be determined by one of ordinary skill in the art. The term also applies to a dose that will induce a particular level of serotonin receptor inhibition in the target cells. The specific dose will vary depending on, for example, the particular compounds chosen, the dosing regimen to be followed, whether it is administered in combination with other agents, timing of administration, the tissue to which it is administered, and the physical delivery system in which it is carried.

The term “selective inhibition” or “selectively inhibit” as applied to a biologically active agent refers to the agent's ability to selectively reduce the target signaling activity as compared to off-target signaling activity, via direct or interact interaction with the target.

The terms “treatment,” “treating,” “palliating,” and “ameliorating” are used interchangeably herein. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compositions can be administered to a patient at risk of developing SSRI-induced adverse outcomes in her pregnancy.

As used herein, a “pharmaceutically acceptable form” of a disclosed compound includes, but is not limited to, pharmaceutically acceptable salts, hydrates, solvates, isomers, prodrugs, and isotopically labeled derivatives of the disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response, and the like, and are commensurate with a reasonable benefit/risk ratio. Pharmaceutically acceptable salts are well known in the art. See, for example, Stahl and Wermuth (Eds.), “Pharmaceutical Salts: Properties, Selection, and Use, 2nd Revised Edition,” 2011 Wiley-VCH, ISBN 978-3906390512.

### Methods to Inhibit Adverse Pregnancy Outcomes Due to SSRIs

Disclosed herein are investigations into the impact of maternal use of SSRIs on the occurrence of intra-uterine growth restriction (IUGR) and preterm birth. Using several in vivo experimental approaches (using as model animals sheep, mice, and mutant mice), it was established that SSRI inhibition of the maternal serotonin transporter (SERT) increases maternal circulating serotonin. The increased serotonin caused by SSRI treatment was the main driver for IUGR, preterm birth, and increased neonatal morbidity/mortality by affecting placental function which compromises fetal development.

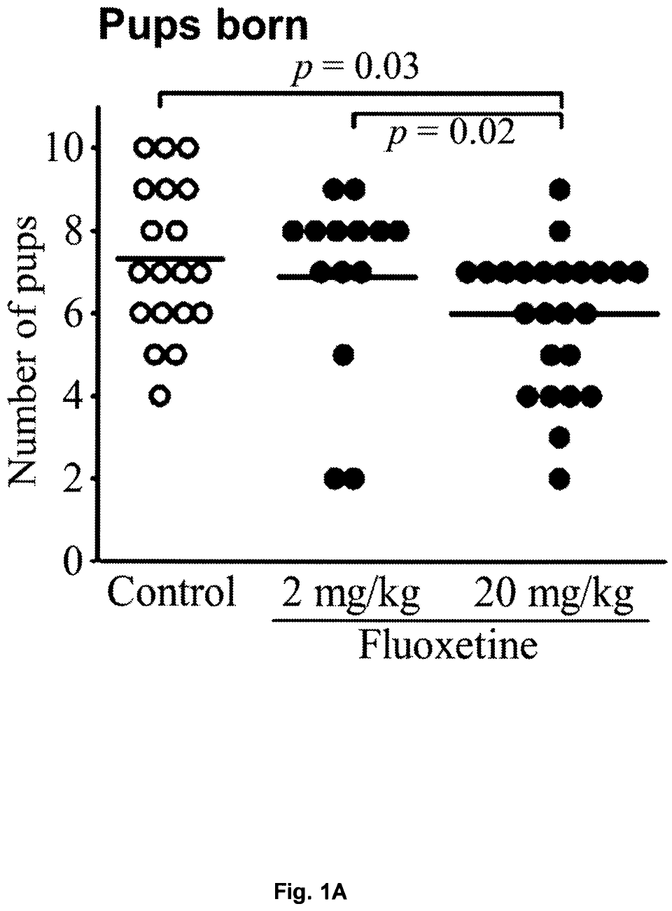

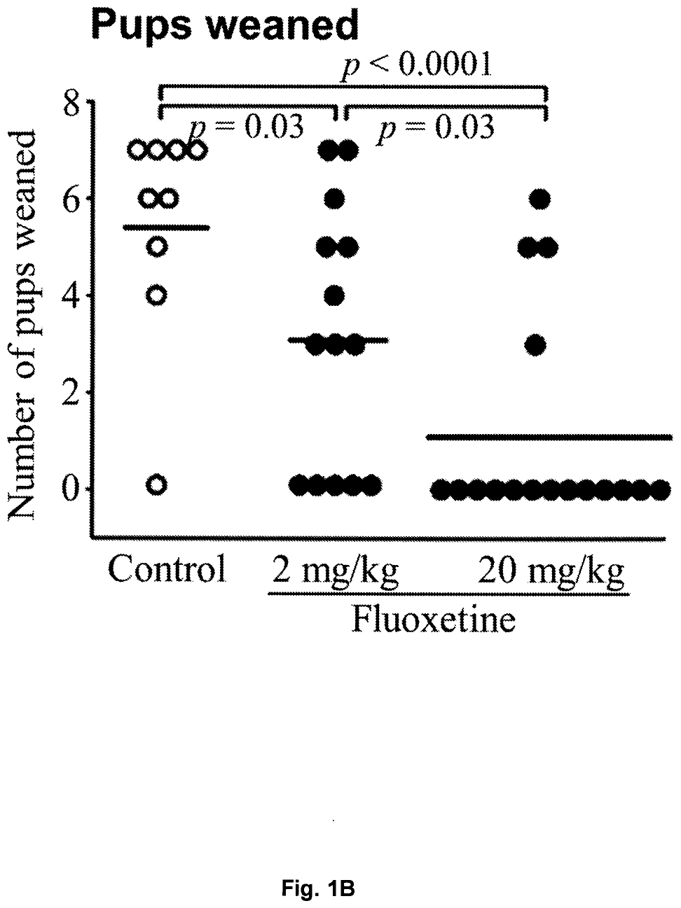

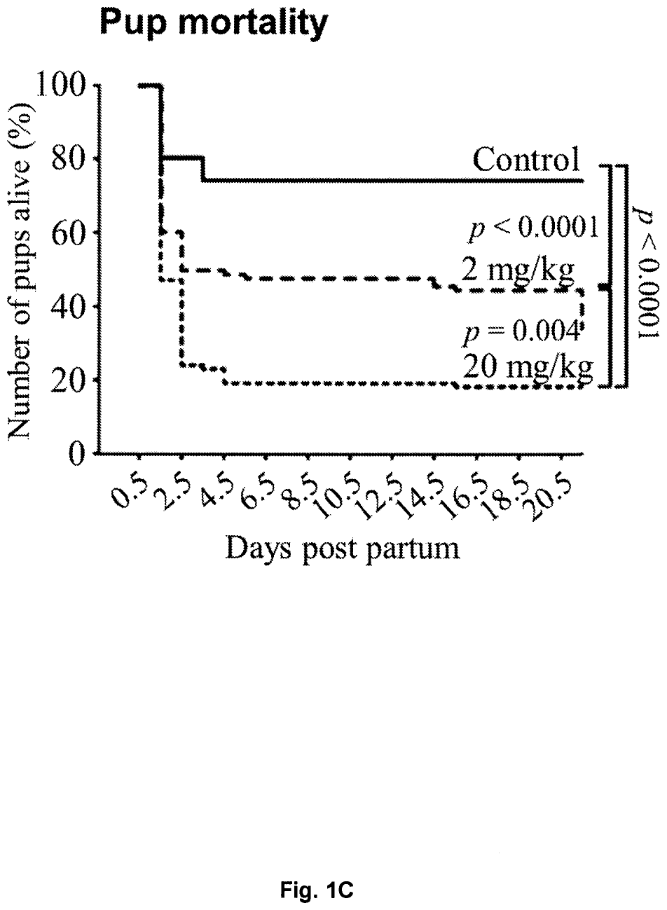

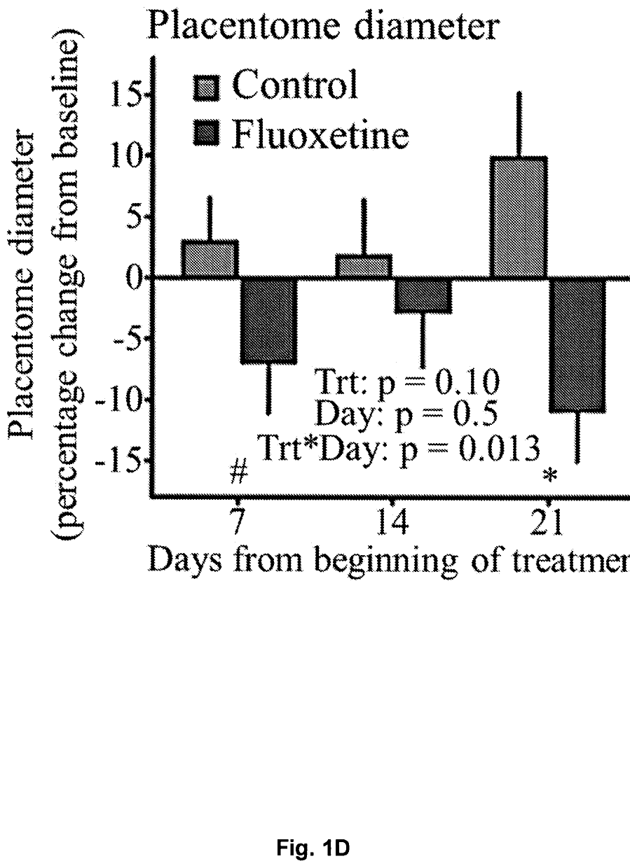

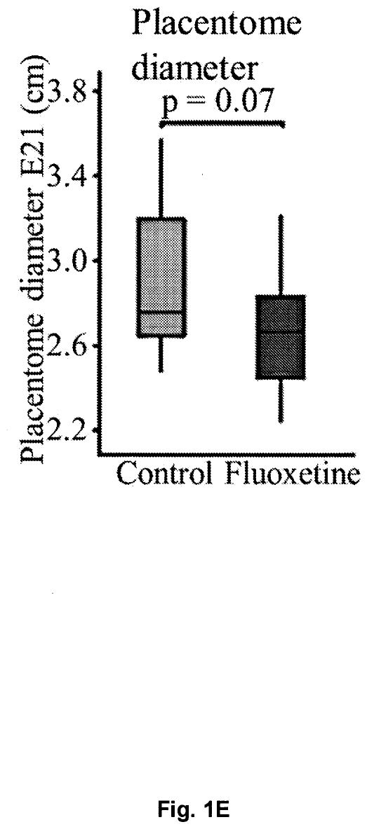

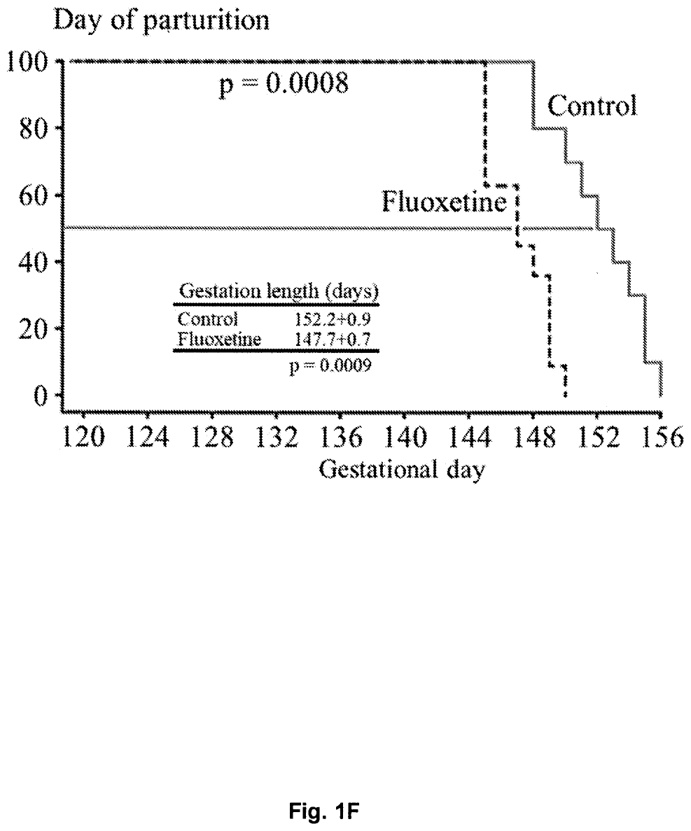

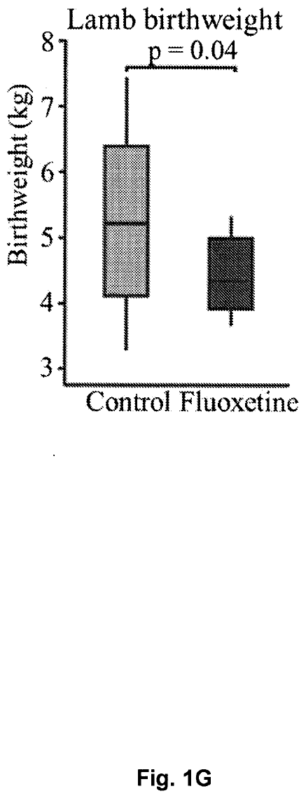

Referring now to FIGS. 1A-IG, this series of figures shows the effects of administering a SSRI (fluoxetine) on pregnancy outcomes in mice and sheep. FIG. 1A depicts the number of pups born to mice that received no SSRI (control) versus a daily dose of 2 mg/kg and 20 mg/kg fluoxetine. As can be seen from the figure, the average number of pups was significantly reduced in the two treatment groups versus the control (p=0.03 as between the control group and the 20 mg/kg group). FIG. 1B shows the effect of the same dosages of fluoxetine (2 mg/kg and 20 mg/kg) on the average number of pups weaned. The results here are quite stark: administering the SSRI had a profound negative impact on the number of mice pups weaned (p=0.03 between controls and the 2 mg/kg cohort and p<0.0001 between controls and the 20 mg/kg cohort). FIG. 1C shows the effect of the same dosages of fluoxetine (2 mg/kg and 20 mg/kg) on mouse pup mortality. Again, the results are quite stark. Administering fluoxetine had a decidedly negative effect on pup mortality—an effect which was dose-dependent (p<0.0001 between controls and the 2 mg/kg cohort and p=0.004 between controls and the 20 mg/kg cohort).

FIGS. 1D through 1G depict similar outcomes in sheep. FIG. 1D depicts placentome diameter (as a percentage of change from a baseline) in control sheep (no SSRI) and a treatment group given SSRI (fluoxetine) over time. The placentome in the treatment group was significantly smaller than in the control group. FIG. 1E shows in isolation the placentome diameter ranges at Day 21 in absolute terms (cm). FIG. 1F shows the days of parturition between the control group and the treatment group. As shown in the inset table in FIG. 1F, average gestational length in the control group was 152.2 days. In the treatment group, the average gestational age was significantly shorter, at 147.7 days (p=0.0009). FIG. 1G compares the lamb birthweights between the treatment group and control group. Again, the lambs born to the treated sheep weighed significantly less than the lambs born to the control group.

Further disclosed herein is a method for alleviating the adverse effects of SSRIs in pregnancy through the co-administration of a serotonin receptor antagonist. It is shown herein that the adverse effects in pregnant mice treated with fluoxetine were prevented by using a serotonin receptor 2A/2C antagonist, ketanserin, to prevent increasing serotonin signaling. Importantly, ketanserin does not affect the antidepressant effects of SSRI. Thus, the method disclosed herein allows women to benefit from the effects of SSRIs in the brain without harming pregnancy outcomes. The method thus inhibits SSRI-induced adverse pregnancy outcomes without ameliorating the psychiatric/psychological benefits of taking an SSRI in the first instance. The method allows pregnant women to benefit from the antidepressant effects of SSRIs without compromising the fetal health of their gestating children.

To investigate the role of ketanserin, an exemplary serotonin receptors 2A and 2C antagonist, on preventing SSRI-induced adverse pregnancy and neonatal outcomes, pregnant mice were treated with SSRI, serotonin, and SSRI/serotonin+ketanserin. Similar findings were observed between SSRI and serotonin groups, consistent with previous reports of the effects of serotonin on pregnancy outcomes: decreased placental weight and size and reduced litter size. Additionally, the studies extended previous findings demonstrating decreased neonatal birthweight and increased mortality. Furthermore, specific histopathological placental alterations associated with increased serotonin were observed-decreased placenta area (total, junctional zone, and labyrinth), increased cell death in the junctional zone and labyrinth, and increased cell turnover in the labyrinth.

Specifically, in experiment 1, pregnant C57Bl/6J mice were treated daily with saline (control; n=11), fluoxetine (one of the most popular SSRI; 2 mg/kg; n=14) or serotonin (5 mg/kg; n=12) from DPC 10.5 until parturition. Serotonin reduced (p<0.0001) maternal weight gain between DPC10.5 and the day before parturition (8.0±0.8 g). Maternal weight gain in fluoxetine-treated dams (10.9±0.9 g) was intermediate, although it was not significantly different from the control (12.5±0.0.5 g). Litter size was decreased (p<0.03) for dams treated with fluoxetine (6.8±0.4) and serotonin (5.2±0.5) compared to the control (8.3±0.4). Neonatal weight on postnatal days 0 and 1 were decreased (p<0.006) for the serotonin group (1.138±0.028 and 1.223±0.028 g) compared to the control (1.271±0.021 and 1.347±0.032 g). Lastly, overall neonatal mortality was greater in the serotonin (32.3%; p<0.0001) and fluoxetine (11.6%; p=0.06) groups compared with the control (4.1%). In experiment 2, pregnant mice were treated as in experiment 1; however, all mice received ketanserin (1 mg/kg) daily one hour before saline (n=6), fluoxetine (n=5) or serotonin (n=5) treatment. Ketanserin prevented the serotonin-induced decrease in maternal weight gain (10.2±0.6 vs 11.4±0.5 g; p>0.1). Additionally, litter size was not different (p>0.1) between serotonin (7.0±0.5) and fluoxetine (7.4±0.6) groups compared with control (7.0±0.3). Neonatal weight was decreased in the serotonin group only on postnatal day 0 (1.197±0.01 vs 1.264±0.01 g; p=0.002). Lastly, overall neonatal mortality was not different for fluoxetine (24.3%; p=0.11) and serotonin (22.8%; p=0.18) groups compared to the control (8.5%). Thus, diminishing the effects of increased circulating serotonin (either due to serotonin or SSRI treatment) using a serotonin receptor antagonist prevents some adverse pregnancy and neonatal outcomes.

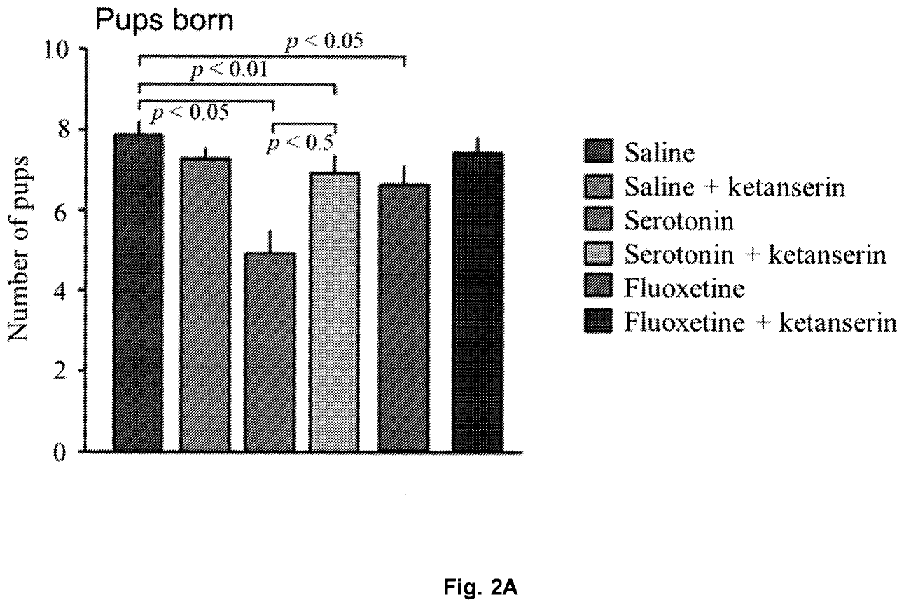

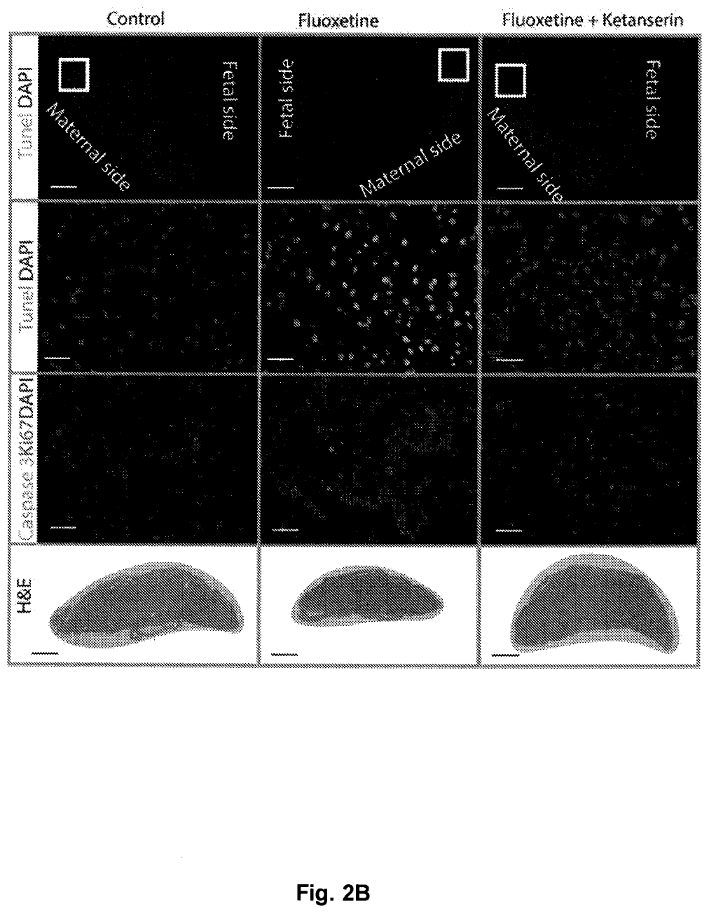

Additionally, FIG. 2A shows the number of mouse pups born in various control and treatment groups (saline, saline+ketanserin (1 mg/kg), serotonin (4 mg/kg), serotonin (4 mg/kg)+ketanserin (1 mg/kg), fluoxetine (2 mg/kg), and fluoxetine (2 mg/kg)+ketanserin (1 mg/kg).) The results here are quite compelling. The control group, which received just saline (far left bar in FIG. 2A) had an average number of pups of just under 8 per litter. This compares extremely favorably with the fluoxetine+ketanserin cohort (far left bar). In contrast, in the serotonin cohort (third bar from left), the number of pups born was significantly reduced, which was also seen in the fluoxetine cohort (second bar from right). FIG. 2B depicts immunostaining for Tunel and caspase 3 (apoptosis) and Ki67 (proliferation) in the placenta (embryonic day 18.5) of mice treated with saline (control), fluoxetine (20 mg/kg), and fluoxetine (20 mg/kg)+ketanserin (1 mg/kg). As seen in FIG. 2B, Tunel-positive apoptotic cells were visualized in the fluoxetine treatment group (central column, second row of FIG. 2B). The placental size was also much reduced in the fluoxetine treatment group (central column, last row of FIG. 2B), while the placental size was not significantly impacted in the fluoxetine+ketanserin treatment group (right column, last row of FIG. 2B)

In short, Ketanserin treatment prevented most placental changes and adverse pregnancy and neonatal outcomes caused by SSRI use. The placental alterations found in the studies described herein are consistent with the reported effects of serotonin reducing blood perfusion to the uterus and placenta in different animal models and in women taking an SSRI during gestation. Furthermore, the fetal and neonatal outcomes are consistent with impaired embryonic development due to placental insufficiency caused by decreased vascular perfusion.

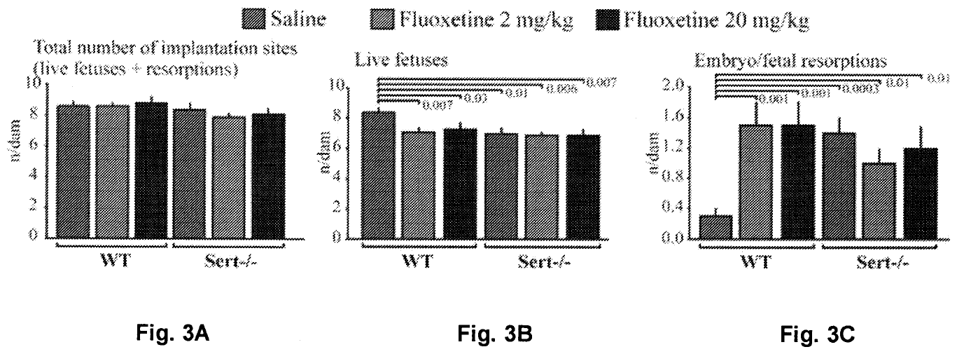

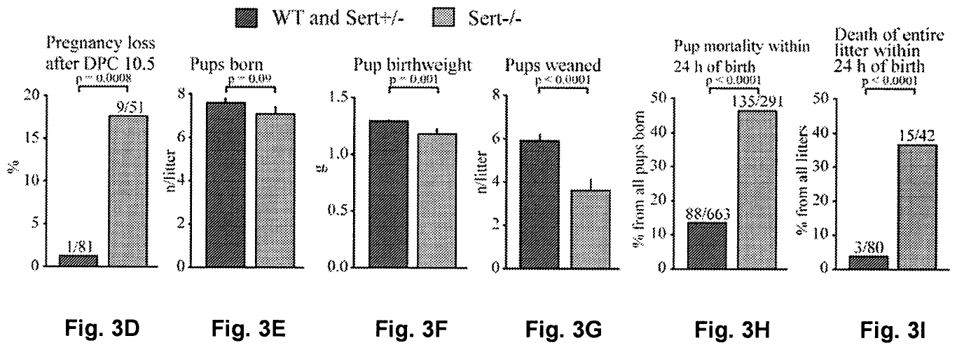

It was also confirmed that the effects of SSRIs, specifically fluoxetine, are mediated through inhibition of SERT, rather than via an off-target pharmacological effect. (The collected results are shown in FIGS. 3A-3I). Here, it was shown that a mutant mouse model with genetic ablation of SERT (i.e., a mouse with no expression of SERT) was not affected by fluoxetine treatment. (In the mutant mice, the 5-HTT gene (htt, slc6a4) has been constitutively inactivated. This can be done using literature methods; see Bengel D, Murphy D L, Andrews A M, Wichems C H, Feltner D, Heils A et al (1998). Mol Pharmacol 53:649-655. HTT-null mutant mice are also commercially available from The Jackson Laboratory, Bar Harbor, Maine.) This shows that SSRIs act through SERT to cause deleterious pregnancy outcomes. In short, the SERT-inhibitory activity of SSRIs and genetic ablation of SERT activity result in similar pregnancy outcomes: increased pregnancy loss, increased number of embryonic resorptions, decreased number of pups born, decreased neonatal weight, and increased neonatal mortality.

FIG. 3A shows the total number of implantation sites (live fetus and resorptions) in wild-type mice (WT) and SERT-null mice (Sert−/−) in control mice (administered saline) and mice administered fluoxetine at 2 mg/kg and 20 mg/kg. Here, small decreases are seen in the Sert−/− mice. FIG. 3B shows the number of live fetuses in the same paired sets of three cohorts: WT control, WT 2 mg/kg SSRI, and WT 20 mg/kg SSRI vs Sert−/− control, Sert−/− 2 mg/kg SSRI, and Sert−/− 20 mg/kg SSRI. Here, the results are more immediately striking. WT mice given fluoxetine at either dose had significantly fewer live fetuses, as did all three Sert−/− cohorts. FIG. 3C presents the most striking results. FIG. 3C shows embryo/fetal resorptions in six groups. The wild-type control group had, by far, the fewest number of embryo/fetal resorptions.

FIG. 3D is a histogram showing pregnancy loss after 10.5 days post coitum (DPC) in wild-type mice and heterozygous Sert+/− mice (as a single cohort) versus SERT-null, Sert−/− mice. The results here are quite striking, with the Sert−/− mice having greatly increased pregnancy loss. FIG. 3E is a histogram showing the number of pups born to the two cohorts. Again, the Sert−/− mice had significantly fewer pups born. FIG. 3F shows pup birthweight. In keeping with the trend, Sert−/− mice had pups who weighed significantly less than pups born to wild-type and heterozygous Sert+/− mice. FIG. 3G is a histogram showing the number of pups weaned in the two cohorts. Again, the Sert−/− mice had significantly fewer pups weaned as compared to wild-type and Sert+/− mice. FIG. 3H records pup mortality within 24 hours in the two groups. The pups of the Sert−/− mice had significantly increased mortality as compared to pups of the wild-type and Sert+/− mice. FIG. 3I is perhaps the most striking set of data—it records deaths of entire liters within 24 hours of birth. Here, the Sert−/− mice fared badly: 15 of 42 litters died entirely within 24 hours of birth. In the wild-type and Sert+/− mice, the corresponding figure was only 3 out of 80.

By performing RNA sequencing of placenta from heterozygous Sert+/− mice vs homozygous Sert−/− knockout mice and their respective offspring, it was confirmed that lack of maternal, but not placental or fetal SERT, is associated with placental changes: more than 2,100 genes were differentially expressed in the placenta when the dam was SERT−/−. Importantly, ketanserin also rescued the neonatal mortality in SERT null mice (49.0 vs 23.8% within 24 hours of birth).

The necessary dosage of the serotonin receptor antagonist for achieving a therapeutically effective treatment can vary with the compound administered, the age and weight of the subject, the general health of the subject, etc. Typically, the dosage ranges from about 0.01 mg/kg bodyweight to about 10 mg/kg body weight.

What is claimed is:

1. A method to inhibit adverse pregnancy outcomes in a pregnant mammal comprising administering to the pregnant mammal a therapeutically effective amount of a serotonin receptor antagonist.

2. The method of claim 1, wherein the serotonin receptor antagonist is a selective serotonin receptor antagonist.

3. The method of claim 1, wherein the serotonin receptor antagonist is a 5-HT_{2A/2C} antagonist.

4. The method of claim 1, wherein the serotonin receptor antagonist is selected from the group consisting of ketanserin, risperidone, trazodone, and nefazodone.

5. The method of claim 1, wherein the pregnant mammal is a human.

6. A method to inhibit adverse pregnancy outcomes in a pregnant mammal comprising administering to a pregnant mammal who is concurrently taking a selective serotonin reuptake inhibitor (SSRI) a therapeutically effective amount of a serotonin receptor antagonist.

7. The method of claim 6, wherein the SSRI is selected from the group consisting of racemic citalopram, escitalopram (S-citalopram), fluoxetine, fluvoxamine, paroxetine, and sertraline.

8. The method of claim 6, wherein the serotonin receptor antagonist is a selective serotonin receptor antagonist.

9. The method of claim 6, wherein the serotonin receptor antagonist is a 5-HT_{2A/2C} antagonist.

10. The method of claim 6, wherein the serotonin receptor antagonist is selected from the group consisting of ketanserin, risperidone, trazodone, and nefazodone.

11. The method of claim 6, wherein the pregnant mammal is a human.
